(19) United States
(12) Patent Application Publication
(10) Pub. No.: US 2017/0240929 A1
(43) Pub. Date: Aug. 24, 2017

(54) Pretreatment of Lignocellulosic Biomasses with Filamentous Fungi for the Production of Bioenergy

(71) Applicants: Institut National de la Recherche Argonomique, Paris (FR); Universite d'Aix Marseille, Marseille (FR); Universite de Reims Champagne Ardenne, Reims (FR)

(72) Inventors: Isabelle Gimbert, Marseille (FR); Sana Raouche, Marseille (FR); Jean-Claude Sigoillot, Six-Fours Les Plages (FR); Simeng Zhou, Marseille (FR); Elsa Rouches, Narbonne (FR); Hélène Carrere, Saint Marcel sur Aude (FR); Estelle Copinet-Legin, Reims (FR)

(21) Appl. No.: 15/514,586

(22) PCT Filed: Oct. 30, 2015
(86) PCT No.: PCT/IB2015/058403

§ 371 Date: Mar. 27, 2017

(30) Foreign Application Priority Data
Oct. 30, 2014 (FR) 14 60472

(51) Int. Cl. C12P 7/10; C12P 5/02; D21C 5/00

(52) U.S. Cl. CPC C12P 7/10; D21C 5/005; C12P 5/023; C12P 2201/00

(57) ABSTRACT

The invention relates to the use of a strain of basidiomycete fungus belonging to the Polyporus brumalis species, for the fungal pretreatment of a lignocellulosic biomass in a solid medium.

The present invention relates to the use of a fungus belonging to the basidiomycetes class for the fungal pretreatment of a lignocellulosic biomass for the production of bioenergy such as ethanol and methane.

In order to anticipate the eventual depletion of fossil energy and contribute to a sustainable environment, the development of renewable energy (water, solar, wind, derived from lignocellulosic biomass) proves to be necessary. Plants or lignocellulosic biomass (LCB) are a substrate of choice for energy production as they are renewable, widely available and rich in sugar (55-75% DM) (Wan & Li, 2012). Furthermore, methanization of lignocellulosic biomass, obtained particularly from agricultural residues, are of increasing interest to the extent that they give rise to limited competition with respect to the use of land for food crops. Yet, this competition is increasingly taken into account in renewable energy development strategy.

Methods for producing bioenergy include the following main steps: 1) pretreatment of the lignocellulosic biomass, such as biological (e.g. fungi or fungal enzymes), physical (e.g. grinding, thermolysis), physicochemical (e.g. thermohydrolysis, steam explosion) or chemical (e.g. dilute acid prehydrolysis, pretreatment under alkaline conditions, use of organic solvents) pretreatment, 2) hydrolysis of the pretreated biomass, such as acid or enzymatic hydrolysis (saccharification step) and 3) fermentation of the hydrolyzed biomass, such as ethanol or methane fermentation.

In energy recovery processed from lignocellulosic biomasses, pretreatment is the preliminary requirement for any hydrolysis or fermentation step. Indeed, it is one of the key steps in which the future output of cellulose hydrolysis and recovery of plant cell wall by-products is determined. The objective thereof is that of breaking down lignocellulose recalcitrant to enzymatic hydrolysis. The lignocellulosic biomass (LCB) consists of cellulose, hemicelluloses and lignin. Lignin hydrolysis is difficult and lignin is poorly biodegradable under anaerobic conditions. Furthermore, in LCB, the bonds and interactions between lignin and other polymers render fermentable sugars (particularly polysaccharides) relatively inaccessible. Chandler et al. (1980) demonstrate that 1% lignin decreases the digestion of organic matter by approximately 3% (Lesteur et al., 2010). For this reason, pretreatments are required to facilitate access to fermentable sugars in LCB (Monlau et al. 2013).

Fungal pretreatment is more particularly intended to selectively degrade lignins under the action of delignification enzymes produced by the fungus during the growth thereof in order to break down the hemilignocellulosic network. This selective delignification of the lignocellulosic substrate preserves the cell wall polysaccharides of the lignocellulosic biomass. This deconstruction facilitates the subsequent steps for saccharification or fermentation of cellulose or hemocellulose by improving the accessibility of the degradation enzymes. The fungal pretreatment step should not be confused with the saccharification step which consists of releasing sugars from cell wall polysaccharides of the lignocellulosic biomass. The sugars released are then subjected to a fermentation step.

An effective fungal pretreatment results in:
selective delignification preserving the holocellulose to enable subsequent conversion to ethanol or biogas and producing aromatic monomers, some of which can be converted during methanization;
low dry matter loss to ensure profitability of the method.

Pretreatments, particularly physical (e.g. grinding) and thermo-chemical (e.g. alkaline, acidic) sometimes represent up to half the energy used for processing the substrate. Enzymatic pretreatments, which require enzyme production and extraction steps, are very costly. The pretreatment step is thus a costly step representing a significant obstacle for the marketing of bioenergy production methods and liable to generate large quantities of waste.

Biological pretreatments involving filamentous fungi represent an alternative to physical, thermo-chemical and enzymatic pretreatments, to the extent that they would enable an economic gain via low energy use or via a lower implementation cost. Of the filamentous fungi, white-rot fungi (essentially basidiomycetes) are the only ones capable of degrading all the components of wood (cellulose, hemicelluloses and lignin) (Cullen et al., 2004). The ability of these filamentous fungi to degrade lignin makes it possible to envisage the industrial use thereof, particularly in methods for producing so-called second-generation (2G) bioenergy, such as biomethane. A distinction can be made between two degradation modes according to the fungi: more or less simultaneous degradation of all wood polymers, and selective delignification such which lignin is preferentially degraded (Kirk et al., 1998). White-rot fungi capable of inducing selective delignification of wood are microorganisms of potential interest for the use thereof in energy recovery processes from lignocellulosic biomasses. A number of studies have related to the use of filamentous fungi in solid state fermentation (SSF) methods on different lignocellulosic substrates and particularly wheat straw. Nevertheless, one drawback of fungal pretreatment is that it requires several weeks to establish a sufficient fungal population to alter the substance significantly.

The effectiveness of fungal pretreatment is evaluated not only by selective delignification of the pretreated lignocellulosic substrate but also by the enzymatic hydrolysis thereof in order to estimate the saccharification potential thereof. The improvement in the digestibility of pretreated lignocellulosic biomasses is generally attributed to delignification and modifications of the physical properties of the substrates: increase in fiber surface porosity; decrease in cellulose crystallinity and alteration of lignin structure which could prevent irreversible cellulase adsorption. In the different studies, the impact of biological pretreatment is analyzed in terms of release of reducing sugars and/or glucose during the step for hydrolysis of the pretreated lignocellulosic substrate. However, it should be noted that it is difficult to perform a comparative analysis of the improvements in hydrolysis reported in the literature due to the disparity of the methods used and particularly the more or less effective cellulosic mixtures used. This methodological disparity is found not only in terms of the operating conditions of the enzymatic hydrolysis of the pretreated biomasses but also in terms of any physicochemical pretreatments associated with the fungal pretreatments.

Furthermore, there is a need to find fungal strains which have high performances for fungal pretreatment.

Within the scope of their research, the Inventors used 5 fungal strains belonging to the basidiomycetes class in a solid state fermentation (SSF) method in order to conduct a biological pretreatment of wheat straw. Wheat straw is an experimental model of lignocellulosic biomass because the composition thereof in organic matter (OM) is representative of herbaceous biomasses (Vassilec et al., 2012). The SSF treatment was conducted at 28° C. for 21 days. The study of the chemical composition of the pretreated wheat straw showed effective delignification with lignin losses between 30 and 50% (m/m). As a general rule, this delignification is accompanied by limited degradation of the cell wall polysaccharides. The Polyporus brumalis_BRFM 985 strain is found to be selective with low cellulose and hemicellulose degradation. The enzymatic hydrolysis of pretreated wheat straw, using commercial cellulosic mixtures, made it possible to obtain increases of up to 85% of the quantities of reducing sugars released from cell wall polysaccharides with the Polyporus brumalis_BRFM 985 strain compared to the non-inoculated control straw. The methane potential tests demonstrated that it was possible to obtain up to 26% additional methane with the Polyporus brumalis_BRFM 985 strain compared to the non-inoculated control straw (NmL CH₄/g of pretreated organic matter). Furthermore, the enzymatic hydrolysis and methanization results demonstrate the effectiveness of the fungal pretreatment of wheat straw for the bioconversion thereof to energy products. The use of the lignolytic Polyporus brumalis_BRFM 985 strain during the lignocellulosic biomass storage period (“active storage”) would thus make it possible to facilitate the subsequent processing of this pretreated lignocellulosic biomass in 2G bioenergy production methods.

The Polyporus brumalis_BRFM 985 strain has been deposited in accordance with the Budapest Treaty dated Oct. 16, 2014 with CNCM (French National Microorganism Culture Collection), 25 rue du Docteur Roux, Paris, under the number CNCM I-4900.

Consequently, the present invention relates to the use of the Polyporus brumalis CNCM I-4900 strain for the pretreatment of lignocellulosic biomass in a solid medium.

The Polyporus brumalis CNCM I-4900 strain can be presented in the form of miscanthus inoculated with said strain. The miscanthus can thus be presented in various forms such as granules, chips, mulch or briquettes of miscanthus, preferably granules. The dimensions of the miscanthus chips can be between 8 and 50 mm.

The term “pretreatment” denotes the total or partial breakdown of the lignocelluloses (delignification) of a lignocellulosic biomass.

The selectivity and effectiveness of the breakdown of lignocellulose can be estimated by measuring, respectively, the recovery of the various cell wall substrates and the quantities of glucose and xylose (derived from xylan, the main hemicellulose in lignocelluloses) released by said lignocellulosic biomass during an enzymatic hydrolysis step.

The “lignocellulosic biomass” essentially consists of cellulose (25 to 55%), hemicellulose (20 to 50%) and lignin (10 to 35%) (the percentages of the different polymers varying according to the lignocellulosic biomass plant resource). The lignocellulosic biomass used according to the present invention includes:
agricultural residues (e.g. wheat or rice straw, stalks),
forest residues (e.g. barks, leaves, branches) from resinous (e.g. pines, spruces) or broadleaf (e.g. eucalyptus, poplar, willow) tree species,
wood processing by-products (e.g. sawdust, scrap),
ligneous or herbaceous plants (e.g. miscanthus, triticale) from dedicated crops,
municipal green waste,
wood waste (e.g. pallets, crates, wood from furnishings),
fermentable fractions of household waste.

The lignocellulosic biomass can consist of fragments of less than 50 cm, preferably between 1 mm and 50 cm.

Advantageously, the lignocellulosic biomass is wheat straw, which can be presented in windrow form.

The pretreatment can be performed during the storage of the lignocellulosic biomass prior to the use thereof in a method for producing bioenergy (e.g. ethanol, methane).

The present invention also relates to a method for producing bioenergy comprising the following steps: i) pretreatment of the lignocellulosic biomass in a solid medium, ii) hydrolysis of the pretreated biomass, such as acid or enzymatic hydrolysis, and iii) fermentation of the hydrolyzed biomass, such as ethanol or methane fermentation, characterized in that said pretreatment step comprises contacting of the Polyporus brumalis CNCM I-4900 strain with said lignocellulosic biomass.

The lignocellulosic biomass used for the implementation of said method is as defined above.

Said pretreatment step comprising contacting of the Polyporus brumalis CNCM I-4900 strain with the lignocellulosic biomass is hereinafter referred to as the fungal pretreatment step.

The Polyporus brumalis CNCM I-4900 strain can be presented in the form of miscanthus inoculated with said strain. The miscanthus can thus be presented in various forms such as granules, chips, mulch or briquettes of miscanthus, preferably granules. The dimensions of the miscanthus chips can be between 8 and 50 mm. The duration of the fungal pretreatment step varies according to the quantity of the Polyporus brumalis CNCM I-4900 strain contacted with the lignocellulosic biomass, the composition of the lignocellulosic biomass, the quantity of the lignocellulosic biomass, the humidity, temperature and aeration conditions of the lignocellulosic biomass, and the result of the pretreatment sought by those skilled in the art. The duration of this fungal pretreatment step can be determined by those skilled in the art based on their general knowledge using routine methods. Advantageously, the duration of the pretreatment step is 5 to 100 days.

The humidity, temperature and aeration conditions of the lignocellulosic biomass during the fungal pretreatment step can also be determined by those skilled in the art based on their general knowledge using routine methods. Advantageously, the humidity is kept constant throughout the duration of the fungal pretreatment step, preferably at a level of 50% to 100%. More advantageously, air is blown into the lignocellulosic biomass, preferably between 0.1 and 3 vvm and/or occasional turnings of the lignocellulosic biomass are performed. More advantageously, the temperature of the lignocellulosic biomass is maintained between 25 and 35° C. for the entire duration of the fungal pretreatment step.

The fungal pretreatment step can be implemented prior, simultaneously or subsequently to a chemical or physicochemical treatment step of said lignocellulosic biomass.

The fungal pretreatment step can also be implemented prior, simultaneously or subsequently to an enzymatic pretreatment step of said lignocellulosic biomass comprising the use of an enzymatic mixture comprising hemicellulases and/or auxiliary degradation enzymes of cell wall polysaccharides. Such enzymatic mixtures are commercially available.

The present invention will be understood more clearly using the supplementary description hereinafter, which refers to the pretreatment of lignocellulosic biomass (wheat straw) with filamentous fungi for the production of bioenergy, along with the appended figures:

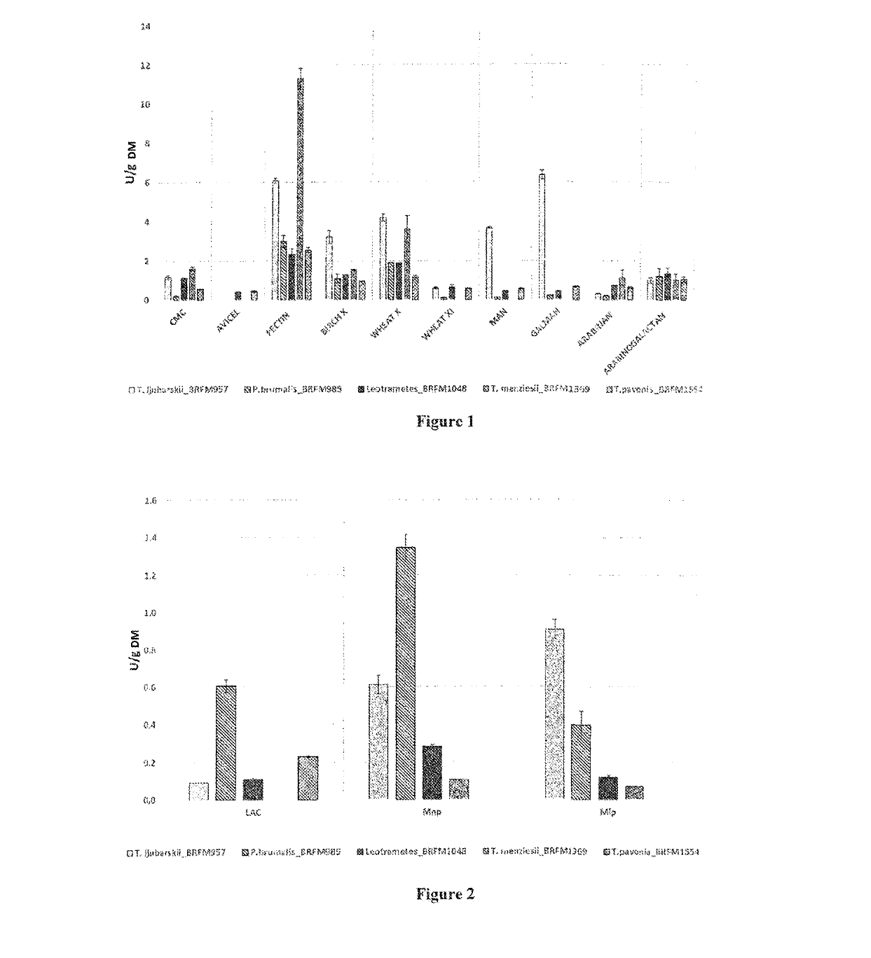

FIG. 1: Glycoside hydrolase activities on complex substrates secreted by filamentous fungi during pretreatment. CMC: Carboxymethyl cellulose; Birch X: Birch xylan; Wheat X: Wheat xylan; Wheat XI: Insoluble wheat xylan; Man: Mannan; GalMan: GalactoMannan.

FIG. 2: Lignolytic activities secreted by filamentous fungi during pretreatment. Lac: laccase; Mnp: Manganese peroxidase; Mip: manganese-independent peroxidase.

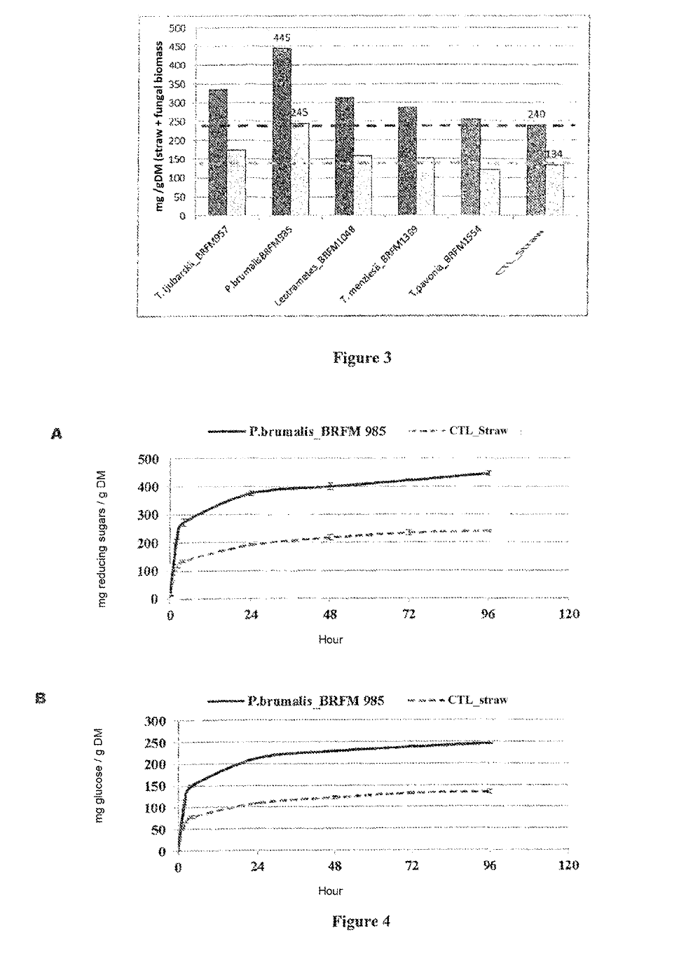

FIG. 3: Impact of fungal strains used for wheat straw pretreatment on quantities of reducing sugars (left bar) and glucose (right bar) released after 96 hours of enzymatic hydrolysis.

FIG. 4: Production kinetics of reducing sugars (A) and glucose (B) during enzymatic hydrolysis of wheat straw pretreated with the P. brumalis strain (BRFM 985). The control wheat straw (CTL_Straw) is non-inoculated wheat straw subjected to the same operating conditions (SSF for 21 days+post-treatments).

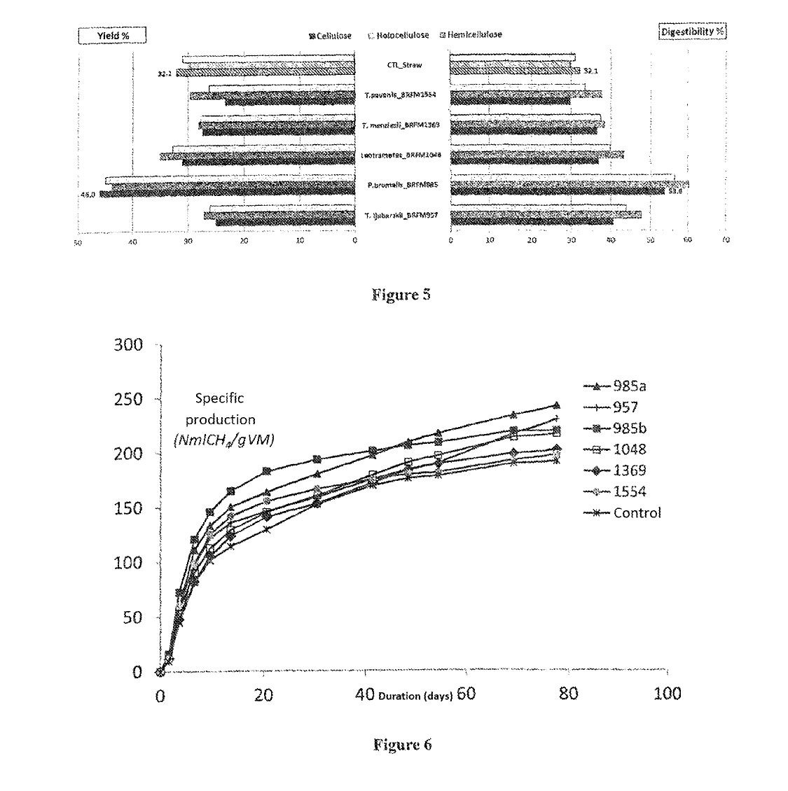

FIG. 5: Digestibility and conversion yields of cell wall polysaccharides of pretreated wheat straw. Cellulose: bottom bar; Holocellulose: top bar; Hemicellulose: middle bar.

FIG. 6: BMP (Biological Methane Potential) of wheat straw in NmL of methane per gram of pretreated VM (volatile matter).

### EXAMPLE 1

### Effect of Fungal Pretreatment on Wheat Straw Composition: Effective Delignification of Biomass

The impact of fungal pretreatments on the lignocellulosic biomass (wheat straw) conducted within the scope of our study was evaluated by determining the contents of the main cell wall constituents (cellulose, hemicelluloses and lignin) and the mass yields.

### Methodologies

### Fungal Strains

Five BRFM (Marseille fungal resource bank) supplied by CIRM (International Center for Microbial Resources, Marseille) were selected on the basis of screening for the potential thereof to be used as a biological pretreatment agent (SSF cultures) for lignocellulosic substrates (wheat straw and miscanthus) in order to improve the subsequent enzymatic hydrolysis thereof:

BRFM 957: Trametes ljubarskii

BRFM 985: Polyporus brumalis CNCM I-4900

BRFM 1048: Leiotrametes sp

BRFM 1369: Trametes menziesii

BRFM 1554: Trametes pavonia

These strains belong to the group of ligninolytic filamentous wood-decay fungi. They belong to the basidiomycetes class, to the Polyporales order and to the Polyporaceae family.

### Culture Conditions

The SSF cultures are produced in glass columns with an effective volume of 250 mL (20 cm in height and 4 cm in diameter). A column contains 20 g of dry matter (DM) of wheat straw, 0.5 g of glucose and 50 mg of diammonium tartrate. Adding a source of carbon and nitrogen in minimum quantities is intended to ensure the initiation of growth of the fungus. Each column is inoculated with 120 mg of DM of mycelium homogenate. The columns are immersed in a thermostatically-controlled tank at 28° C. for 21 days. The initial water retention of the straw is 90%. A humidity-saturated upward air flow is controlled at 120 ml/min by a ball flow meter.

For each of the strains, the SSF cultures were tripled and the pretreated wheat straw obtained pooled and homogenized to supply batches of substrates in sufficient quantities for the analyses thereof. Non-inoculated wheat straw is treated under the same conditions and as such represents the control wheat straw.

### Determination of Dry Matter Controls and Mass Yields of Pretreated Wheat Straw

The dry matter contents and mass yields of the pretreated wheat straw are determined using the pretreated wheat straw with or without washing with water.

The dry matter contents are determined by gravimetry by measuring the weight loss obtained after drying the solid in an oven at 105° C. to a constant weight (after 48 hours). Based on the dry matter, mass yields (%) are calculated according to the following expression:

Mass yield (%)=Dry matter after treatment/Dry matter before treatment×100

### Determination of Cell Wall Polysaccharide and Lignin Composition of Pretreated Wheat Straw

The determination of the cell wall polysaccharide and lignin composition of the biomass is based on a two-step acid hydrolysis according to the operating protocol described by the NREL (National Renewable Energy Laboratory). The first step consists of hydrolysis at 30° C. in the presence of 72% sulfuric acid for 1 hour. The acid is then diluted to 4% and the second acid hydrolysis step is conducted in an autoclave at 120° C. for 1 hour. The hydrolysate is then filtered and the supernatant is analyzed by enzyme assay (RTU glucose kit, Biomérieux) and colorimetric assay (dinitrosalicylic acid method) in order to the cellulose and hemicellulose contents. The lignin is partially solubilized during hydrolysis (acid-soluble lignin). The solid residue obtained after hydrolysis thereby contains acid-insoluble lignin and ash.

### Results

The filamentous fungi cultured on wheat straw draw the metabolites and energy required for the growth thereof from this substrate. Furthermore, the fungal metabolism results in the release of saccharide fractions (oligosaccharides and monomeric sugars) and non-assimilated lignin fragments and which are found in the washing water of the pretreated wheat straw.

This results in dry matter losses, the values of which are given in table 1 below.

TABLE 1
Mass yields of pretreated wheat straw

| | | BRFM No. | | | | | |
|---|---|---|---|---|---|---|---|
| | | 957 | 985 | 1048 | 1369 | 1554 | Control^{c} |
| | | Strain | | | | | |
| | | T. ljubarskii | P. brumalis | Leiotrametes sp | T. menziesii | T. pavonia | — |
| Without | Mass yield ^{a} | 68.7 ± 2.5 | 83.3 ± 1.2 | 82.1 ± 2.4 | 76.5 ± 2.6 | 81.8 ± 1.0 | 96.5 ± 0.7 |
| wash | (%) | | | | | | |
| | Overall mass | 71.2 | 86.3 | 85.1 | 79.3 | 84.8 | 100.0 |
| | yield ^{b} (%) | | | | | | |
| With | Mass yield ^{a} | 52.9 ± 1.2 | 68.9 ± 1.7 | 71.5 ± 1.4 | 66.1 ± 1.7 | 70.2 ± 1.0 | 88.0 ± 1.0 |
| wash | (%) | | | | | | |
| | Overall mass | 60.0 | 78.2 | 81.2 | 75.0 | 79.7 | 100.0 |
| | yield ^{b} (%) | | | | | | |

^{a} The final mass of wheat straw determined after SSF is referenced to the initial wheat straw mass before SSF.
^{b} Corresponds to the mass yield with respect to the control corresponding to 100%.
^{c}Non-inoculated wheat straw treated with SSF.

As a general rule, mass yields of approximately 80% are obtained after 21 days of SSF. The greatest loss of matter was caused by T. ljubarskii (40%).

The composition of the wheat straw after fungal treatment was analyzed (see table 2 hereinafter). The specific degradations of each of the main cell wall constituents, expressed as a percentage of the initial content thereof were determined (see table 3 hereinafter).

TABLE 2
Cellulose, hemicellulose and lignin
compositions of the various samples as a % (g of
constituent/100 g Dry Matter of pretreated wheat straw)

| BRFM | Cellulose % | Hemicelluloses % | Lignin % | Sum % |
|---|---|---|---|---|
| 957 | 38.5 ± 0.1 | 29.8 ± 0.3 | 18.1 ± 0.2 | 86.4 |
| 985 | 41.0 ± 0.2 | 29.2 ± 0.3 | 17.1 ± 0.1 | 87.3 |
| 1048 | 38.6 ± 0.4 | 31.4 ± 0.4 | 18.2 ± 1.1 | 88.2 |
| 1369 | 37.3 ± 0.5 | 30.5 ± 0.3 | 19.3 ± 0.2 | 87.1 |
| 1554 | 36.8 ± 0.4 | 31.0 ± 0.8 | 18.6 ± 0.4 | 86.4 |
| Control ^{a} | 37.5 ± 0.9 | 31.4 ± 0.3 | 21.9 ± 1.0 | 90.8 |

^{a} Non-inoculated wheat straw treated with SSF.

TABLE 3
Cellulose, hemicelluloses and lignin yields of the various samples

| | Mass | Cellulose | | Hemicelluloses | | Lignin | |
|---|---|---|---|---|---|---|---|
| BRFM | yield ^{a} % | % ^{b} | % loss ^{c} | % ^{b} | % loss ^{c} | % ^{b} | % loss ^{c} |
| 957 | 60.0 | 23.1 | 38.4 | 17.9 | 43.1 | 10.8 | 50.5 |
| 985 | 78.2 | 32.1 | 14.5 | 22.9 | 27.3 | 13.4 | 39.0 |
| 1048 | 81.2 | 31.4 | 16.3 | 25.5 | 18.9 | 14.8 | 32.5 |
| 1369 | 75.0 | 28.0 | 25.2 | 22.9 | 27.2 | 14.5 | 33.9 |
| 1554 | 79.7 | 29.3 | 21.8 | 24.7 | 21.5 | 14.8 | 32.4 |
| Control ^{d} | 100.0 | 37.5 | — | 31.4 | 0.0 | 21.9 | — |

^{a} Overall mass yields (see Table 1).
^{b} Composition expressed as a % (g of constituent/100 g Dry Matter of pretreated wheat straw). It accounts for the mass yield of the fungal pretreatment specified in the preceding column. For the calculation thereof: mass yield × % constituent_{table 2}
^{c} The loss percentages were determined with respect to the composition of non-inoculated control straw (the values determined for each constituent of the control straw make up 100%)
^{d} Non-inoculated wheat straw

All the filamentous fungi studied delignify wheat straw effectively. Of these, P. brumalis_BRFM 985 and Leiotrametes sp_BRFM 1048 are the most effective for selective straw delignification (33-39% delignification) while preserving cellulose (84-85% preservation). Moreover, it is interesting to note that T. menziesii_BRFM 1369, T. pavonia_BRFM 1554 and Leiotrametes sp_BRFM 1048 degrade cellulose and hemicelluloses with similar loss rates (22-25% and 21-27% respectively, for BRFM 1369 and BRFM 1554; 16% and 19% respectively, for BRFM 1048) whereas P. brumalis_BRFM 985 preferentially use hemicelluloses as a carbon source thereby preserving most of the cellulose (27% degradation of hemicelluloses and 14% degradation of cellulose). The T. ljubarskii_BRFM 957 strain appears to simultaneously degrade cellulose, hemicelluloses and lignin (38%, 43% and 50% respectively).

These data on the composition of the plant cell walls associated with the mass yields of fungal pretreatments provide information on the quantity of glucose and xylose (main compound of wheat straw hemicelluloses) that can theoretically be released during enzymatic hydrolysis. The duration of the fungal pretreatment of the wheat straw set at 21 days appears to be suitable for the majority of strains since the holocellulose and dry matter losses remain reasonable and compatible with bioenergy production methods. For T. ljubarskii_BRFM 957, the significant degradation of lignocellulose associated with a high dry matter loss is suggestive of a probably excessively long pretreatment time under the culture conditions used.

Each pretreated wheat straw was subjected to enzymatic hydrolysis and the conversion yield of polysaccharides to fermentable sugars (digestibility) was calculated.

### EXAMPLE 2

### Biological Characterization of Fungal Pretreatment: Lignocelluloytic Enzymes Secreted

The breakdown of lignocelluloses is the result of the secretion by the filamentous fungi of complex lignocellulolytic enzyme systems including hydrolytic and oxidative activities acting synergistically on the different cell wall polymers.

### Methodologies

The different enzyme activities were determined using aqueous extracts (5% (m of wheat straw/v of water) consistency; 1 hr; 4° C.; under stirring) of pretreated wheat straw at an SSF column outlet. These activities were expressed in Enzyme units per gram of dry matter of pretreated wheat straw.

### Glycoside Hydrolase (GH) Activities

The enzyme activities on carboxymethylcellulose (CMC), microcrystalline cellulose (avicel PH-101), xylans, pectins, mannan, galactomannan, arabinan and arabinogalactan were measured using the dinitrosalicylic acid method using glucose as a standard.

A unit of enzyme activity was defined as the quantity of enzyme releasing 1 μmol of reducing sugars per minute.

### Lignolytic Activities

The lignolytic activities measured are laccase, peroxidases and cellobiose dehydrogenase.

The laccase activity is determined by monitoring the oxidation of ABTS (2,2′-azino-bis(3-ethylbenzothiazoline-6-sulfonic acid)).

The overall peroxidase activity including manganese peroxidase (MnP) and the manganese-independent peroxidase (MiP) activity is determined by monitoring the oxidation of a solution of DMP (2,6-dimethyloxyphenol) in the presence of MnSO4, H₂O₂ and sodium fluoride. On the basis of this activity, it is possible to distinguish:
the manganese-independent peroxidase (MiP) activity which is determined by monitoring the oxidation of a solution of DMP (2,6-dimethyloxyphenol) in the presence of only H₂O₂;
the manganese peroxidase (MnP) activity which is determined by monitoring the oxidation of a solution of DMP (2,6-dimethyloxyphenol) in the presence of MnSO4 and H₂O₂. This activity is calculated by subtracting the MiP activity from the overall peroxidase activity.

The cellobiose dehydrogenase activity corresponds to the rate of reduction of 2,6-dichlorophenol-indophenol (DCPIP) in the presence of cellobiose and sodium fluoride.

The ligninolytic activities are expressed in U/g DM where a unit of enzyme activity has been defined as the quantity of enzyme oxidizing 1 μmol of substrate per minute.

### Results

The filamentous fungi studied produce a diversity of hydrolases (FIG. 1) and ligninolytic enzymes (FIG. 2). It is interesting to note that none of the strains produces CDH (cellobiose dehydrogenase).

Of the strains tested, P. brumalis_BRFM 985 is the strain which produces the lowest GH activities and the greatest ligninolytic activities. This enzymatic profile corroborates selective delignification of wheat straw by this fungus. On the other hand, T. ljubarskii_BRFM 957 secretes the most GH activities. This result is consistent with the significant degradation of holocellulose observed for this fungus.

### EXAMPLE 3

### Effect of Fungal Pretreatments on Enzymatic Hydrolysis: Improvement of Hydrolysis Kinetics and Yields of Pretreated Straw

The purpose of the fungal pretreatment is to break down lignocellulose in order to render the cell wall polysaccharides more accessible to hydrolysis and thereby increase the fermentable sugar yields. The effectiveness of the fungal pretreatment was evaluated by determining the digestibilities and conversion yields of the cell wall polysaccharides of the pretreated wheat straw. These were calculated on the basis of the measurements of the quantities of reducing sugars and glucose released upon the hydrolysis thereof by commercial mixtures of GC220 cellulases of Trichoderma reesei and SP188 of Aspergillus niger.

### Methodologies

The enzymatic hydrolyses are carried out using wheat straw pretreated and treated with sodium hydroxide in a TORNADO reactor (Tornado™ Overhead Stirring System, Radleys Discovery Technologies, United Kingdom). It was demonstrated that the alkaline treatment conducted under mild conditions improves the enzymatic digestibility of the pretreated substrate. It is suggested that this treatment may help remove the mycelium from the fiber surface which could have a negative effect on the subsequent enzymatic step. The lack of washing of the lignocellulosic biomass between the alkaline treatment and enzymatic hydrolysis steps makes it possible to limit losses and ensure the reliability of results.

### Mild Alkaline Treatment

The alkaline treatment conditions are as follows:
Duration: 1 hour
Temperature: 50° C.
Pretreated wheat straw consistency: 6% (m/m)
Sodium hydroxide concentration: 0.12% (m/V) 30 mM equivalent, 2% (m/m)
pH=12

### Hydrolysis Tests

The pretreated wheat straw, washed with water and subjected to the sodium hydroxide treatment, are hydrolyzed with commercial mixtures of cellulases of T. reesei and A. niger. The hydrolysis conditions are as follows:
Duration: 96 hours
Temperature: 50° C.
Consistency: 3% (m/m) in 50 mM Citrate Phosphate buffer, pH 4.4

The decrease in the consistency of the wheat straw suspension, from 6% (alkaline treatment) to 3% by adding buffer makes it possible to attain and maintain the reaction pH at 4.8.
GC220 cellulases: 12 U/g DM
β-glucosidase SP188: 60 U/mg DM
Antibiotics: 0.15 ml/ml tetracycline and 0.04 mg/ml cycloheximide

The addition of antibiotics makes it possible to prevent any microbial contamination.
Stirring: 500 rpm.

### Assay of Sugars Released

The measurement of the quantities of glucose released during hydrolysis was conducted by enzyme assay (RTU glucose kit, Biomérieux), using a glucose standard curve (2 to 10 mM). The reducing sugars released were measured by the dinitrosalicylic acid method using glucose as a standard (2 to 10 mM).

### Determination of Digestibilities, Crystallinity of Cellulose and Conversion Yields of Cell Wall Polysaccharides of Pretreated Straw

The digestibilities of cellulose and hemicelluloses were determined according to the following expression:

$$Digestibility ( \% ) = \frac{g of glc or xyl released / g of pretreated straw}{g of glc or xyl released / g of pretreated straw} \times 100$$

This data item expresses the yield of the enzymatic hydrolysis step.

X-ray measurements were made with an analytical X-ray diffractometer (Philips Analytical), using Cu Ka radiation at k=0.1540 nm (40 kV, 40 mA). The measurements were made on powder compacted into small sheets. The X-ray diffraction (XRD) data were collected for an angle range 2θ between 5° and 50° with an interval of 0.02°. The crystallinity index (CrI) of cellulose was expressed as a percentage. The equation used for calculating the CrI is:

$$CrI = \frac{I 002 - Iam}{I 002} \times 100$$

where I₀₀₂ corresponds to the peak intensity read on the meter, for an angle 2θ of 22° and Iₐₘ corresponds to the peak intensity for an angle 2θ of 16°. I₀₀₂-Iₐₘ corresponds to the peak intensity of crystalline cellulose and I₀₀₂ is the total peak intensity of cellulose after subtracting the background noise measured without cellulose. Crystalline cellulose was determined using the equation:

Crystalline cellulose_{XRD}(% DM)=CrI×Cellulose NREL (% DM)

The conversion yields of cellulose and hemicelluloses make it possible to account for the dry matter losses caused by the fungal pretreatments. They are expressed as a percentage and are calculated according to the following expression:

$$Conversion yield ( \% ) = \frac{( g of glc or xyl released / g of pretreated straw ) * mass yield}{g of glc or xyl released / g of non \text{-} inoculated control straw} \times 100$$

This data item expresses the yield of the method (pretreatment+enzymatic hydrolysis).

### Results

All the pretreated wheat straw hydrolyzed by cellulases of T. reesei and A. niger made it possible to obtain quantities of reducing sugars released greater than that of the control straw with increases of up to 85%. Increases of 12% to 83% of the quantity of glucose released from pretreated wheat straw were observed (FIG. 3). The pretreatment of wheat straw with the P. brumalis_BRFM 985 strain proved to be the most effective for improving the enzymatic hydrolysis giving rise to increases of 85% and 83% of reducing sugars and glucose released, respectively.

The hydrolysis kinetics of the wheat straw pretreated with the most effective strain, P. brumalis_BRFM 985, are shown in FIG. 4.

The digestibilities and conversion yields of the different cell wall polysaccharides of the pretreated wheat straw into fermentable sugars were determined. The results are represented in FIG. 5.

For the majority of the strains tested, the enzymatic digestibilities of the pretreated wheat straw compared to that of the control straw increased, in particular with Leotrametes_BRFM 1048, P. brumalis_BRFM 985 and T. ljubarskii_BRFM 957 (FIG. 5).

Cellulose crystallinity is recognized as being a parameter limiting enzymatic attack (Hendriks & Zeeman, 2009). The crystallinity measurements of the straw pretreated with the strains which give rise to improvements in enzymatic hydrolysis (T. ljubarskii BRFM 957 and P. brumalis BRFM 985) as well as the control straw are reported in table 4 hereinafter. The results show a significant reduction in crystallinity during the pretreatment with these two strains.

TABLE 4
Measurement of crystallinity by XRD and
crystalline cellulose content

| | Crystallinity | Crystalline |
|---|---|---|
| | index CrI | cellulose |
| | (% DM Cellulose) | (% DM) |
| T. ljubarskii | 26.85 | 14.45 |
| BRFM 957 | | |
| P. brumalis BRFM | 36.27 | 10.28 |
| 985 | | |
| Control^{a} | 51.09 | 20.84 |

^{a}non-inoculated wheat straw

Moreover, two of these fungi, Leotrametes_BRFM 1048 and P. brumalis_BRFM 985, gave rise to significant increases in the conversion yields of the different cell wall compounds. The significant delignification rate of the wheat straw pretreated with T. ljubarskii_BRFM 957 (approximately 50%) is in correlation with the improvement in the digestibility thereof. However, the significant loss of dry matter caused by this pretreatment has an adverse effect on the conversion yields of lignocellulose. The best conversion yields obtained from wheat straw pretreated with P. brumalis_BRFM 985 attain values of 46%, 43.7% and 45% conversion of cellulose, hemicelluloses and holocellulose, respectively.

### EXAMPLE 4

### Effect of Fungal Pretreatments on Methanization: Improvement of Biochemical Methane Potential of Pretreated Straw

The methanization reaction induces the conversion of the organic matter into biogas mainly consisting of methane and carbon dioxide. The use of complex microbial consortia enables the conversion of the majority of organic compounds (sugars, proteins, fats) into biogas with the exception of lignin. Under certain conditions, aromatic compounds from the degradation of lignin can be converted into biogas. In the case of the pretreated straw, the holocelluloses accessible to microorganisms as well as the fungal biomass are converted into biogas. The pretreated samples were methanized without washing so as not to lose the biogas obtained from labile sugars and the fungal biomass.

### Methodologies

### Determination of Volatile Matter (VM) Contents

The volatile matter contents (representing the organic matter) are determined by gravimetry (APHA standard method, 1998). This consists of the weight loss obtained by combustion (4 hrs at 550° C.) on a previously dried sample (passed for 48 hrs at 105° C.)

### Measurement of Biochemical Methane Potential (BMP)

The BMP tests are conducted under favorable conditions for methanization and indicate the maximum quantity of methane that can be obtained for these substrates. They were conducted with the control straw and the pretreated strew, unwashed and freeze-dried, as well as on a fungal biomass (BRFM 1554) cultured in liquid medium, washed with water (to remove traces of culture medium) and freeze-dried. It is noted that two samples pretreated with the BRFM 985 strain were measured for BMP.

The 600 mL BMP flasks have an effective volume of 400 mL, which contains the substrate (1.3 g DM/flask), a methanizer inoculum (3 g VM/L), water, macro- and micro-elements along with a bicarbonate buffer in order to ensure optimal methanization conditions. The flasks are placed at 36° C. under stirring and the biogas production was monitored by measuring the pressure (pressure gauge), until the end of production (plateau phase), in triplicate. The measurement of the biogas composition was performed for each pressure reading with a micro-gas chromatography analysis: Varian GC-CP4900 (injector at 100° C., capillary columns at 30° C., carrier gas: helium). The volumes of gas are expressed in Normal milliliters (NmL). They consist of the volumes obtained under normal temperature and pressure conditions (0° C., 1 atm).

### Results

The pretreated samples have a BMP greater than that of the control, which reflects the improvement in sugar accessibility particularly due to delignification (see FIG. 6 and table 5 hereinafter). In particular, the BRFM 957 and BRFM 985 strains resulting in the greatest delignification rates also results in the highest methane potentials (approximately 230 NmL CH4/g pretreated VM). Moreover, it is interesting to note that white rot is readily biodegraded: the BRFM 1554 strain has a BMP of 256±60 NmL CH4/g VM, which is greater than that of the control straw (192±9 NmL CH4/g VM). As such, even if there are material losses, a portion of the lignocellulosic biomass is converted into fungal biomass. In other words, the straw is converted into more readily biodegradable biomass.

TABLE 5
BMP and standard deviation of the different
samples

| | BMP | | BMP | |
|---|---|---|---|---|
| | (NmL | | (NmL | |
| | CH4/g | | CH4/g | |
| | pretreated | Standard | initial | Standard |
| BRFM | VM) | deviation | DM) | deviation |
| 985a ^{a} | 242 | 15 | 198 | 12 |
| 985b | 220 | 2 | 181 | 1 |
| 957 ^{a} | 230 | 9 | 153 | 6 |
| 1048 ^{a} | 217 | 12 | 185 | 10 |
| 1369 ^{a} | 202 | 6 | 166 | 5 |
| 1554 ^{a} | 198 | 1 | 159 | 7 |
| Control | 192 | 9 | 184 | 9 |

^{a} Non-definitive results (end-of-product plateau not reached)

The improvement in methane production is to be compared to the loss of matter caused. As such, the results were expressed in NmL/g initial DM (Table 5), i.e. per g DM before SSF (determined by means of the mass yield). Accounting for the loss of matter obtained for the culture conditions used, only a few strains can increase the BMP of the straw (BRFM 1048 and BRFM 985). The BRFM 957 strain makes it possible to obtain a significant BMP when this is expressed with respect to the VM, which reflects the significant delignification. On the other hand, it is the most mediocre when the result is expressed with respect to the initial DM, which reflects the significant DM losses occurring during SSF.

### EXAMPLE 5

### Pretreatment of 2 Metric Tons of Wheat Straw with the Polyporus Brumalis CNCM I-4900 Strain

### Preparation of Inocula

Primary Inoculum (Production of Fungal Biomass with Liquid Fermentation)

The P. brumalis CNCM I-4900 strain is inoculated on malt-based agar medium and incubated for 5 to 10 days at 25-35° C.

A preculture is produced in Roux flasks containing 200 ml of malt-based medium. Each flask is inoculated with 5 disks of mycelium (5 mm in diameter) sampled on agar. After 7-10 days of growth at 25-35° C. and protected from light, the mycelium is harvested and ground using a mill (Ultra Turrax).

The homogenate is used to inoculate a bioreactor having an effective volume of 50 liters in order to produce the primary inoculum. The inoculation rate is between 0.5 and 5% m/v. The liquid medium for biomass production medium in a fermenter consists of malt extract and yeast extract. The aeration is between 0.1 and 3 vvm and the stirring between 50 and 200 rpm. The temperature of the culture is maintained between 25 and 35° C.

After 2 to 10 days, a maximum biomass production is attained. It is harvested and then used as a primary inoculum for the production of miscanthus colonized by the P. brumalis CNCM I-4900 fungus forming the secondary inoculum. Miscanthus can thereby be produced in different forms such as granules, chips, mulch or briquettes of miscanthus.

### Secondary Inoculum (Production of Colonized Miscanthus by SSF).

Miscanthus, for example in the form of granules, chips, straw or briquettes, serves as a nutrient substrate for the P. brumalis CNCM I-4900 fungal strain.

The miscanthus is packaged in bags or trays and humidified to a level of 50 to 300%.

This miscanthus is inoculated between 0.1% and 2% m/m with primary inoculum.

The aeration of the fungal culture is promoted by packaging the inoculated miscanthus in containers below the maximum filling capacity thereof and by the closure thereof with a filtering device.

The bags or trays are periodically stirred so as to promote the homogeneity of the colonization of the substrate.

The incubation lasts from 4 to 15 days, preferably between 4 and 7 days, at a temperature between 25 and 35° C.

The inoculated miscanthus can be used extemporaneously, after storage between 4° C. and 15°, after freeze-drying or any other preservation means.

If necessary, this step is repeated before the inoculation of the straw, in order to increase the quantity of inoculum available.

### Fungal Pretreatment of Wheat Straw

The straw is inoculated with the secondary inoculum at an inoculation rate between 5 and 50%, preferentially between 20 and 40% and humidified to a level of 50 to 100%.

The inoculated wheat straw is stored in trays equipped with forced aeration equipment. This device enables the air to be blown between 0.1 and 3 vvm. Occasional turnings of the lignocellulosic matter promote the aeration of the culture and consequently the fungal growth.

The temperature is maintained between 25 and 35° C.

The humidity is kept constant throughout the duration of the pretreatment.

The duration of the pretreatment is between 7 and 90 days.

### REFERENCES

Chandler, J. A., & Jewell, W. J. (1980). Predicting methane fermentation biodegradability (p. 234). Solar Energy Research Institute.

Cullen D, Kersten P.J. (2004). Enzymology and molecular biology of lignin degradation. In: R. Brambl, G. A. Marzluf, The mycota iii: Biochemistry and molecular biology—second edition. Berlin-Heidelberg: Springer-Verlag.

Hendriks A T W M., Zeeman G. (2009). Pretreatments to enhance the digestibility of lignocellulosic biomass. Bioresource Technology. 100, 10-8.

Kirk T. K., Cullen D. (1998). Enzymology and molecular genetics of wood degradation by white-rot fungi. in: R. A. Young, M. Akhtar, Environmentally friendly technologies for the pulp and paper industry (pp. 273-307). New York: John Wiley & Sons.

Lesteur, M., et al. (2010). Process Biochemistry, 45(4), 431-440 (review).

Monlau, F., et al. (2013). Critical Reviews in Environmental Science and Technology, 43(3), 260-322.

Vassilev, S. V, et al. (2012). Fuel, 94, 1-33.

Wan, C., & Li, Y. (2012). Biotechnology Advances, 30(6). 1447-57.

1. (canceled)

2. A method for producing bioenergy comprising pretreatment of a lignocellulosic biomass in a solid medium, hydrolysis of the pretreated biomass and fermentation of the hydrolyzed biomass, wherein said pretreatment comprises contacting the lignocellulosic biomass with Polyporus brumalis CNCM I-4900 strain.

3. The method of claim 2, wherein-the lignocellulosic biomass is selected from the group consisting of agricultural residues, forest residues, wood processing by-products, ligneous or herbaceous plants, municipal green waste, wood waste and fermentable fractions of household waste.

4. The method of claim 3, wherein the lignocellulosic biomass is wheat straw.

5. The method of claim 2, wherein the Polyporus brumalis CNCM I-4900 strain is present in the form of miscanthus inoculated with said strain.

6. The method of claim 5, wherein the miscanthus is in the form of granules, chips, mulch or briquettes of miscanthus inoculated with said strain.

7. The method of claim 2, wherein the duration of the pretreatment is 5 to 100 days.

8. The method of claim 2, wherein the humidity of the lignocellulosic biomass is kept constant throughout the duration of the pretreatment.

9. The method of claim 2, wherein air is blown into the lignocellulosic biomass and/or occasional turnings of the lignocellulosic biomass are performed during the pretreatment.

10. The method of claim 2, wherein the temperature of the lignocellulosic biomass is maintained between 25 and 35° C. for the entire duration of the pretreatment.
